(19) United States
(12) Patent Application Publication
(10) Pub. No.: US 2026/0155238 A1
(43) Pub. Date: Jun. 4, 2026

(54) MULTIMODAL IMAGE REGISTRATION

(71) Applicant: Carl Zeiss Meditec, Inc., Dublin, CA (US)

(72) Inventor: Homayoun Bagherinia, Oakland, CA (US)

(73) Assignee: Carl Zeiss Meditec, Inc., Dublin, CA (US)

(21) Appl. No.: 19/408,694

(22) Filed: Dec. 4, 2025

Related U.S. Application Data
(60) Provisional application No. 63/727,815, filed on Dec. 4, 2024.

(51) Int. Cl. G16H 30/20; G06T 7/33

(52) U.S. Cl. CPC G16H 30/20; G06T 7/337; G06T 2207/10101; G06T 2207/30041

(57) ABSTRACT

Systems, methods and devices are disclosed for registering different ophthalmic images of different modalities including different fundus imager modalities and OCT/OCTA modalities.

### CROSS-REFERENCE TO RELATED APPLICATIONS

This application claims priority to, and the benefit of, Provisional Patent Application No. 63/727,815, filed Dec. 4, 2024, and titled “MULTIMODAL IMAGE REGISTRATION,” which is incorporated by reference herein in its entirety for all purposes.

### FIELD

The present disclosure is generally directed to a method and system for fundus image registration. More specifically, the disclosure is directed to using multimodal and multichannel data for fundus registration.

### BACKGROUND

Retinal fundus imaging allows one to diagnose many eye diseases such as hypertension or diabetes.

Another use of fundus imaging is the imaging of blood flow (e.g., angiography) in the eye. For example, indocyanine green angiography (ICGA) is an ophthalmologist-performed test that provides information about the circulation of the retina and choroid. ICGA can image the choroidal circulation and its abnormalities because indocyanine green (ICG) can be visualized through the retinal pigment epithelium (RPE), lipid exudates, and serosanguineous fluid. ICGA is a well-established technique for detecting and distinguishing age-related macular degeneration (AMD) from other chorioretinal diseases.

Another modality of fundus imaging is color fundus photography (CFP), which is a key imaging modality for detecting and grading AMD. Image registration between ICGA and multi-color fundus images is important for the joint diagnosis of retinal lesions.

The ICG test provides information about the choroid, choroidal neovascular membrane (CNVM) or other causes of subretinal or sub-macular hemorrhage. Fundus photography captures the images of the retina, optic nerve head, macula, retinal blood vessels, choroid, and the vitreous. Similarly, ICGA fundus images capture choroidal vessels, whereas superficial and deeper retinal vessels are more visible in color fundus images.

A fundus and optical coherence tomography (OCT) combo device provides different fundus imaging modalities, such as ICGA and color fundus imaging, as well as 3D OCT scans and other OCT imaging modalities. For example, the projection of the choroidal layer (CH) of an OCT scan provides a 2D enface image with choroidal vessel patterns similar to that of an ICGA fundus image. Similarly, the projection of the superficial retinal layer (SRL) and deeper retinal layer (DRL) of an OCT scan provides a 2D enface image with similar vessel patterns as in a color fundus image.

Registering images from these (and other) image modalities is crucial to correctly compare pathology changes in a pair of images. A part of any image registration process is the identification of matching landmarks between an image pair. Finding matching landmarks across both images may not be possible. Therefore, a different solution may be used.

Some challenges to traditional fundus image registration include:
1) Landmark matching can be a difficult problem due to changes in pathology over time observed in between when two fundus images in an image pair were taken, possible complicated transformation between two images, and quality of the images;
2) The registration of two images acquired from different imaging technologies (e.g., different imaging modalities) such as OCT, Infrared reflectance (IR), and color fundus is challenging.
3) Transformation models (e.g., projective, or non-linear transformation) between two images can be complicated due to hardware and patient fixation differences.

Landmark-based registration can work if:
1) A sufficient number of matched landmarks (e.g., feature points) are found.
2) Well distributed landmarks matches are found across the two images being registered to each other.
3) A suitable transformation model is selected. Generally, the transformation model is selected prior to applying the image registration algorithm, so a suitable selection is not guaranteed.

Another challenge would be if there is an unknown (image) transformation between two images being registered, especially if the transformation appears to be an unknown or nonlinear transformation.

A few robust approaches used for the detection and matching of features (e.g., feature points) include SIFT (Scale Invariant Feature Transform) and SURF (Speeded up Robust Feature), which are invariant to scale, rotation, translation, illumination, and blur. However, these methods may not be robust enough when there is a nonlinear transformation between images.

Another approach is to use nonlinear registration, which uses a nonlinear registration model and a single iteration.

In summary, prior art fundus image registration approaches typically use nonlinear registration model directly, use a single iteration, and use known a transformation model.

The systems and methods improve the probability of successful (e.g., retinal) image registration, particularly of images taken over a span of time over which changes in imaged subject (e.g., retina) may have occurred.

The systems and methods improve image registration of different imaging modalities.

The systems and methods automate the selection of a suitable global transformation model for use in an image registration process.

### SUMMARY

The systems and methods for fundus image registration (or other type of image registration) may incorporate multiple features. A first feature is to use multichannel landmark matches (e.g., landmarks from multiple corresponding channel types, e.g., color channels) of two fundus images with varying number of channels. For example, fundus image registration may use multichannel matches of landmarks detected in corresponding channels (e.g. red channel of a first image to red channel of a second image, green channel of the first image to green channel of the second image, etc.). Using multiple channel landmark matching may increase the number of matched landmarks and their distribution (e.g., well-distributed landmarks preferably span the entirety or majority of an overlap area of two images being registered to each other). In this manner, landmarks are detected in pairs of matched channels. In various embodiments, landmarks can be detected in a pair of matched and unmatched channels, e.g., the XY positions (e.g., in a cartesian plane) of the landmark matches may be used for registration. In a third feature, the number of channels can be different in each image being registered. In a fourth feature, the image modality of the images being registered to each other can be different.

In various embodiments:
1) Landmarks are detected in two fundus images with varying (and optionally unequal) number of channels;
2) Landmarks can be detected in a pair of matched and unmatched channels;
3) The number of channels can be different in each image being registered; and
4) The image modality of the two images being registered can be different.

In various embodiments:
1) two images may be registered in an iterative process;
2) at each iteration, a distorted image is warped closer to a reference image;
3) a projective transform is used as a registration model at each iteration;
4) at each iteration, it is expected that more feature matches are found between the two images being registered as the distorted image is warped closer to the reference image;
5) a simpler feature matcher such as template matching using normalized cross-correlation may be used (non-scale and rotational invariant), e.g., at each iteration since the distortion between the two images being registered is being reduced with each iteration and the matched feature points between iterations can be cumulative for purposes of final image registration.

Various embodiments may include the following steps:
1) register two images in an iterative process;
2) nonlinear registration is replaced by a projective transform at each iteration;
3) the nonlinear relationship between the two images being registered is reduced at each iteration; and
4) a simpler (characteristic) feature matcher (feature matching algorithm) such as template matching using normalized cross-correlation can replace a more complicated feature matcher.

As discussed above, image transformation can be an important part of an image registration process in general, and fundus image registration in particular. Various embodiments may incorporate a method for automatically finding an appropriate global transformation for a pair of images being registered. Herein, a solution is presented to automatically find a suitable global transformation between a pair of images:
1) an objective is to search for a global transformation model between a pair of images;
2) global transformations, such as rigid, affine, and/or projective, are used;
3) the transformation model that minimizes an objective function is selected as the transformation model to be used;
4) the objective function is derived from flow velocity between the pair of registered images;
5) the flow velocity is calculated using optical flow;
6) the method is also applicable for local nonlinear registration between the pair of images;
7) the method can be used for registration quality evaluation.

Herein is also presented a solution to address the registration problem across multiple imaging modalities (e.g., 2D OCT choroidal vessel patterns, 2D OCT vessel pattern images, ICGA images, color fundus photography, ICG images, single channel (e.g., infrared) or multi-channel (e.g., color) fundus photography, etc.). For example,
1) to register an ICGA image to a color fundus image,
2) the ICGA image can be registered to a first OCT enface image (e.g., choroidal slab) (note that an OCT choroidal slab enface image provides information similar to that of ICGA),
3) the color fundus image can be registered to a second image modality of the same 3D OCT data, such as a second, different enface image (e.g., defined from the superficial+deeper retinal layers, or the whole retina layers), note that (A) an OCT retinal layer enface image provides information similar that of a fundus image and that (B) since both OCT enface image modalities are generated from the same 3D OCT data slab, they are inherently registered to each other,
4) These two consecutive image transformations (2 and 3) can be used to transform the ICGA image to the color fundus image by means they being registered to different enface images of the same 3D OCT data.

Thus, contrary to the typical approach of attempting a direct registration approach from an ICGA image to color fundus image, which can pose difficulties, the present approach provides an indirect registration process by using two different OCT enface image modalities (e.g., two different OCT enface images of different retinal layers) in the registration process. Stated more simply, ICGA image and color fundus image are indirectly registered to each other by means of two intermediate enface images generated from a common 3D OCT scan (e. g, an OCT cube scan).

Several publications may be cited or referred to herein to facilitate the understanding of the systems and methods. All publications cited or referred to herein, are hereby incorporated herein in their entirety by reference for all purposes.

The various embodiments disclosed herein are only examples, and the scope of this disclosure is not limited to them. Any embodiment feature mentioned in one claim category, e.g. system, can be claimed in another claim category, e.g. method, as well. The dependencies or references back in the attached claims are chosen for formal reasons only. However, any subject matter resulting from a deliberate reference back to any previous claims can be claimed as well, so that any combination of claims and the features thereof are disclosed and can be claimed regardless of the dependencies chosen in the attached claims.

### BRIEF DESCRIPTION OF THE DRAWINGS

In the drawings wherein like reference symbols/characters refer to like parts:

FIGS. 1-3 show an example registration of a 3-channel color fundus image (e.g., comprised of a red channel, a green channel and a blue channel) being registered to a 1-channel Infrared (IR) image. In particular, FIG. 1 shows the red channel (of the color fundus image) being registered to the IR image with detected matching landmarks shown (indicated by circles). FIG. 2 shows the green channel registered to the IR image with detected matching landmarks, and FIG. 3 shows blue channel registered to the IR image with detected matching landmarks, in accordance with various embodiments.

FIG. 4 shows the exemplary resultant registration of the color fundus image to the IR image using landmarks detected in the pairwise images of FIGS. 1-3, in accordance with various embodiments.

FIG. 5 shows samples image in a first iteration of an iterative multimodal fundus image registration method, in accordance with various embodiments.

FIG. 6 shows sample images in a second iteration of the iterative multimodal fundus image registration method, in accordance with various embodiments.

FIG. 7 shows a sample image in a third iteration of the iterative multimodal fundus image registration method, in accordance with various embodiments.

FIG. 8 shows the exemplary final registration after a fourth iteration of the iterative multimodal fundus image registration method, in accordance with various embodiments.

FIG. 9 is an example that shows the flow velocity magnitude for different global transformations, including a reference fundus image (left) and the moving fundus image (right), in accordance with various embodiments.

FIG. 10 represents the exemplary flow velocity between the reference and the moving image, in accordance with various embodiments.

FIG. 11 shows the exemplary flow velocity for the rigid (left) and projective (right) transform, in accordance with various embodiments.

FIG. 12 shows the exemplary OCTA choroidal slab (e.g., enface image of the OCTA choroidal slab) (left) and ICGA fundus image (right) with overlayed matching points, in accordance with various embodiments.

FIG. 13 shows the exemplary registered images (ICGA image to choroidal slab (e.g., enface image)) overlayed on top of each other with matching points after registration, in accordance with various embodiments.

FIG. 14 shows the exemplary OCTA superficial slab (en face image) (left) and color fundus image (right, shown as a gray scale image) with overlayed matching points (shown as circles), in accordance with various embodiments.

FIG. 15 shows the exemplary registered images (OCTA superficial slab to color fundus) overlayed on top of each other with matching points after registration, in accordance with various embodiments.

FIG. 16 shows the exemplary registered images (registered ICGA image (see step 3) to the color fundus image) overlayed on top of each other, in accordance with various embodiments.

FIG. 17 illustrates an example of a slit scanning ophthalmic system for imaging a fundus, in accordance with various embodiments.

FIG. 18 illustrates an exemplary generalized frequency domain optical coherence tomography system used to collect 3D image data of the eye, in accordance with various embodiments.

FIG. 19 shows an exemplary OCT B-scan image of a normal retina of a human eye, and illustratively identifies various canonical retinal layers and boundaries, in accordance with various embodiments.

FIG. 20 shows an example of an en face vasculature image, in accordance with various embodiments.

FIG. 21 shows an exemplary B-scan of a vasculature (OCTA) image, in accordance with various embodiments.

FIG. 22 illustrates an example computer system (or computing device or computer), in accordance with various embodiments.

### DETAILED DESCRIPTION

The present (fundus) image registration method may use multiple image modalities (i.e., multimodal) and multiple (optionally different) image channels. As an example, the following steps summarize a multimodal registration method for registering a 3-channel image (e.g., a color fundus image including a red channel, green channel, and blue channel) to a 1-channel image (e.g., a single IR fundus image):
1) Use a pair of images (e.g. red-channel of the color fundus image and IR (infrared) image);
2) Select one of the images as the reference image;
3) Find landmarks (e.g., characteristic features) in the reference image by random sampling the reference image or using a feature finding algorithm to find a sufficient number (e.g., a predefined number) of landmarks;
4) Find corresponding reference landmarks in other image using a feature matching/finding algorithm (e.g. template matching);
5) Repeat step 1) to 4) for a different pairs of images (e.g. green-channel and/or blue-channel of the color fundus image and the IR image); and
6) Use all landmark matches detected in all pairs of images for (a cumulative) landmark-based registration. An example of a landmark based registration includes:
a. use RANSAC (random sample consensus) or exhaustive search method to select a subset of best landmark matches (e.g., matches having the highest confidence);
b. use a suitable transformation (e.g. rigid, affine, non-linear);
c. register the color fundus image to the IR image, or vice versa.

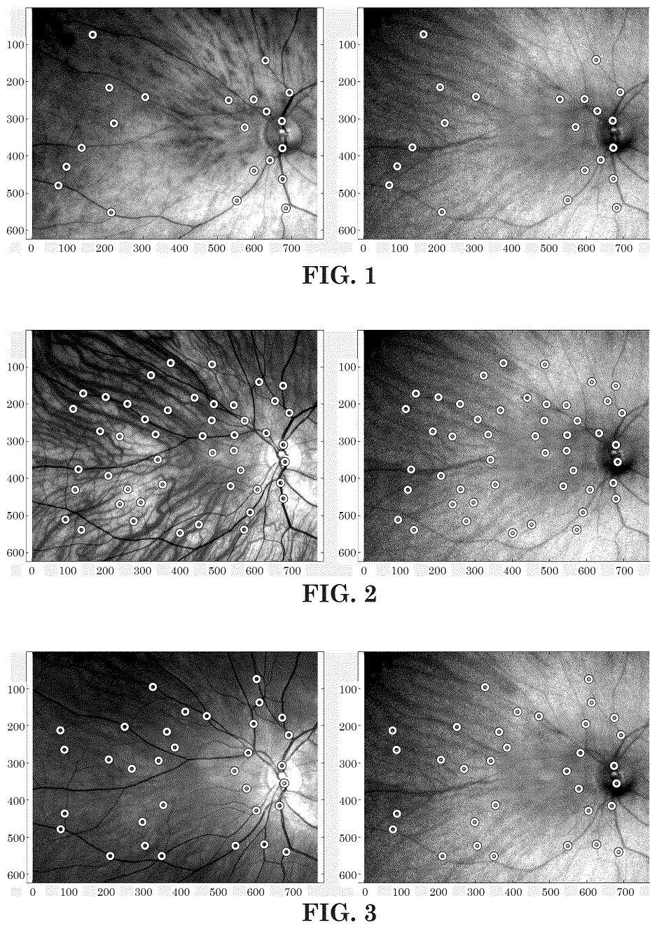

FIGS. 1-3 show an example registration of a 3-channel color fundus image (e.g., comprised of a red channel, a green channel and a blue channel) to a 1-channel Infrared (IR) image. In particular, FIG. 1 shows the red channel (image) 11R (of the color fundus image) paired with the IR image 13 with detected matching landmarks shown (indicated by circles). FIG. 2 shows the green channel (image) 11G paired with the IR image 13 with detected matching landmarks (indicated by circles), and FIG. 3 shows blue channel (image) 11B paired with the IR image with detected matching landmarks (indicated by circles).

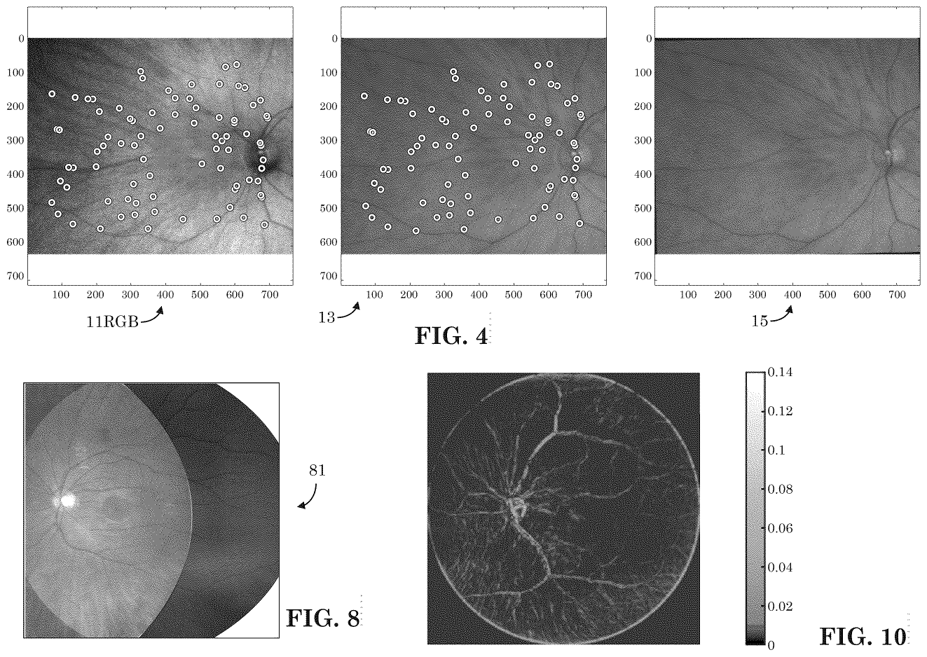

FIG. 4 shows the registration of the color fundus image 11RGB to the IR image 13 using landmarks detected in pairwise images (as illustrated in FIGS. 1-3). Optionally, all the matched landmarks or the best landmarks (e.g., landmarks that meet a predefined confidence level) may be used in the registration of color fundus image 11RGB to the IR image 13. In the present example, a sufficient number of landmarks were found, and the landmarks are well distributed in this example (e.g., the landmarks span a majority of the overlap area of the color fundus image 11RGB and the IR image 13). The resultant registration 15 of color fundus image 11RGB to the IR image 13 is also shown.

Note that additional channels could be generated by:
1) a linear/nonlinear combination of two or more channels of each multichannel image, and/or
2) a linear/nonlinear combination of two or more processed channels (e.g. edge enhanced image/gradient image) of each multichannel image.

The above method can be applied to any number of channels per image.

Another method of fundus image registration that may be used singularly or in combination with other embodiments is herein termed Iterative Multimodal Fundus Image Registration. This approach incorporates an iterative image registration method to an image registration process. An example of an iterative image registration method applied to a color fundus image and an IR fundus image may be summarized as follows:
1) Use a pair of images (e.g. green-channel of the color fundus image and IR fundus image);
2) Designate one of the two images as a reference and other as a distorted image;
3) Find landmarks in the reference image, such as by random sampling the reference image or by using a feature finding algorithm to find sufficient number of landmarks;
4) Initialize the current projective transformation (e.g., matrix T) with the identity matrix, which results in a distorted image. As it would be understood by one versed in the art, a projective transformation (also known as a homography or projectivity) is a mathematical operation that maps points and lines in a projective space to other points and lines while preserving certain fundamental properties related to incidence and perspective, and a projective transformation matrix is an invertible matrix that defines a mapping between two projective spaces, such as between two images. This type of transformation is also known as a homography. As it is also known in the art, an identity matrix is generally a square matrix that has ones on its main diagonal and zeros everywhere else, and may act as the multiplicative identity for matrices.
5) Find corresponding reference landmarks in the distorted image using a feature matching/finding algorithm (e.g. template matching);
6) Use all landmark matches for landmark-based registration, which may include:
   a. use RANSAC (random sample consensus) or other (preferably exhaustive) search method to select a subset of best landmark matches;
   b. find/define a projective transformation matrix T0;
7) Update the current projective transformation matrix T by multiplying to the projective transformation matrix T0;
8) Apply matrix T to the original distorted image to create a new distorted image to be used in step 5) in a next iteration;
9) Repeat step 5 to 9 until the optimization / algorithm is converged.

Note that the projective transformation in step 6 (b) can be replaced with a simpler transformation (e.g. rigid or affine) for the first, or the first few (e.g., 2-5) iterations.

Steps 4 to 9 above describe a mathematical optimization problem. A few objective functions could be considered for this problem:
convergence of T0 to near identity matrix
the error between a previous (e.g., immediately preceding) and a current transformed distorted image (or processed image such as vessel image or gradient image) is smaller than a threshold;
the error between previous and current transformed distorted image regions (or processed image such as vessel image or gradient image) is smaller than a threshold;
the error between previous and current transformed distorted image landmarks is smaller than a threshold;
a combination of above objective functions (each selected objective function is a term).

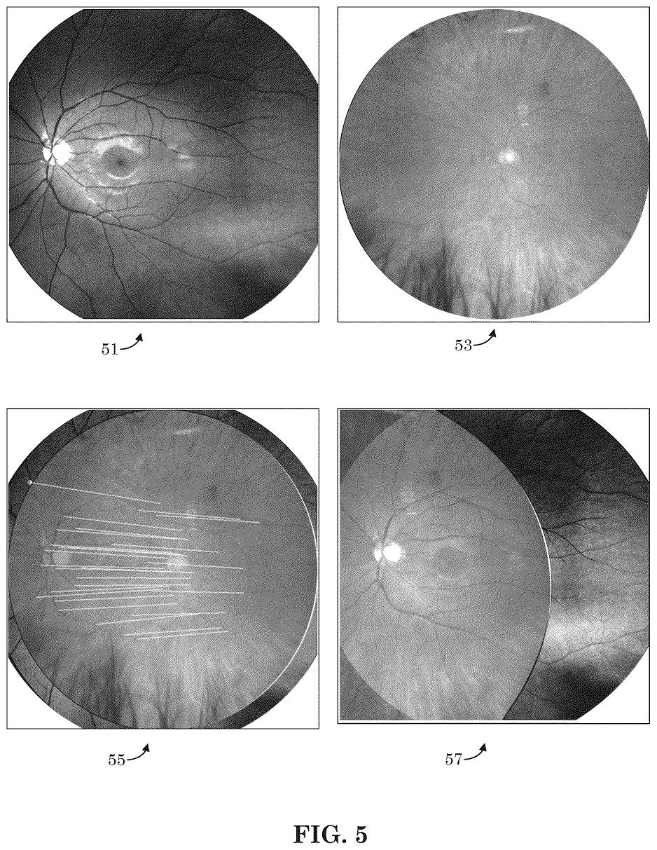

FIG. 5 shows a 1st iteration: green channel of the reference image 51 (top-left), distorted IR image 53 (top-right), overlay of the reference and distorted image before registration with matching landmarks 55 (bottom-left), and overlay of the reference and distorted image after registration 57 (bottom-right).

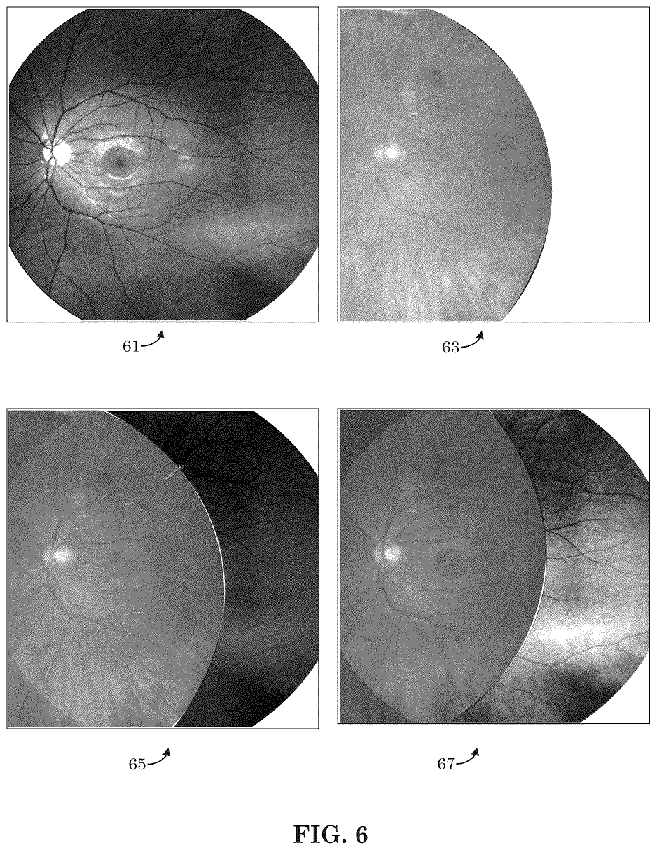

FIG. 6 shows a 2nd Iteration: green channel of the reference image 61 (top-left), transformed distorted IR image 63 (top-right), overlay of the reference and distorted image before registration with matching landmarks 65 (bottom-left), and overlay of the reference and distorted image after registration 67 (bottom-right)

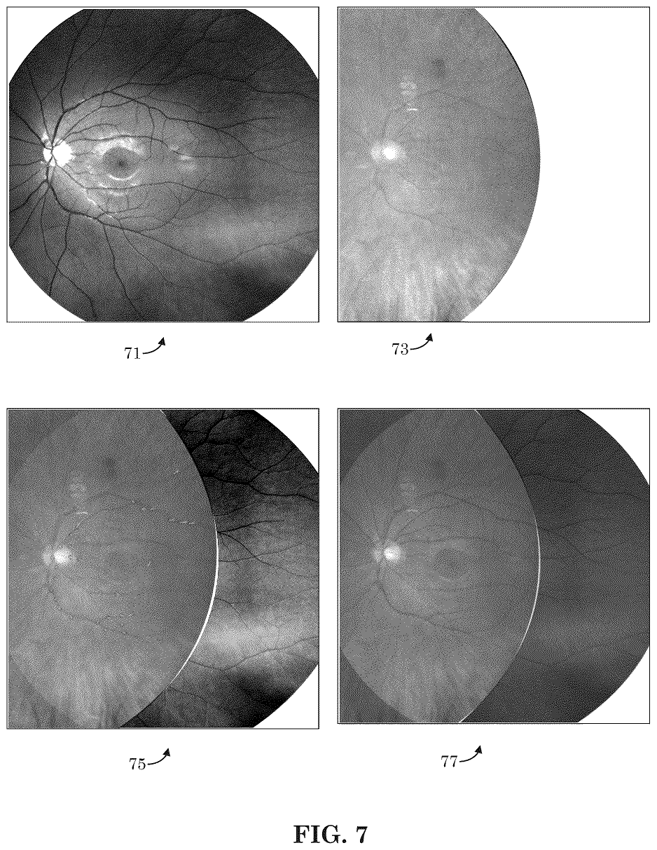

FIG. 7 shows a 3rd Iteration: green channel of the reference image 71 (top-left), transformed distorted IR image 73 (top-right), overlay of the reference and distorted image before registration with matching landmarks 75 (bottom-left), and overlay of the reference and distorted image after registration 77 (bottom-right).

FIG. 8 shows a final registration result 81 after the 4th iteration.

As discussed above, selecting a suitable global transformation model can help the performance of the above image registration methods/processes. A method of global transformation estimation for fundus image registration is now provided.

In various embodiments, the systems and methods may search for a global transformation model between a pair of images. The global transformations may include rigid, affine, and/or projective transformation, or other suitable global transformations. The transformation model that minimizes an objective function is selected as the transformation model for the image registration processes being used. The objective function is derived from flow velocity between a pair of registered images. The flow velocity is calculated using optical flow.

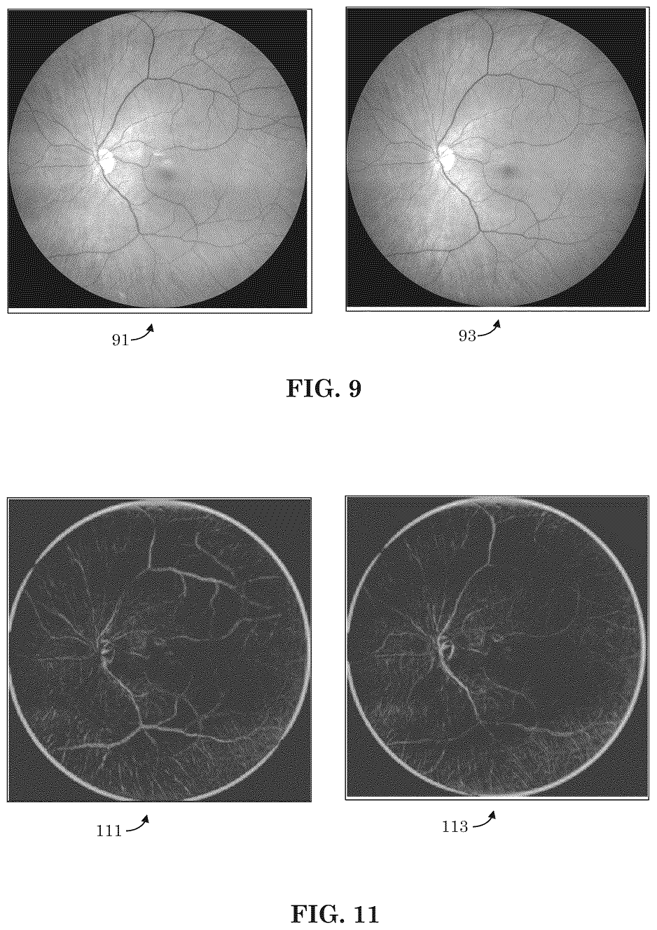

FIG. 9 is an example that shows the flow velocity magnitude for different global transformations. The left image 91 is the reference fundus image, and the right image 93 is the moving fundus image.

FIG. 10 represents the flow velocity between the reference and the moving image. The velocity magnitude is seen clearly at large vessels.

FIG. 11 shows the flow velocity for the rigid 111 (left) and projective 113 (right) transform. The flow velocity is smaller in local image regions using projective transform.

The objective function can be calculated by mean or sum of flow velocities along the large vessels or in the EDTRS gird centered at the fovea or ONH. Any other local regions can be considered depending on the region of interest. The vessel image can be created from known algorithms in the literatures. The objective function can be adapted based on the region of interest. For instance, the weighted sum of the EDTRS grid sectors can define the objective. The global transform that minimizes the objective function is selected as the transformation model.

This method is also applicable for local nonlinear registration between a pair of images. This method can also serve as a visualization tool and an automatic image registration evaluation tool. For instance, if the flow velocity in a region or entire image is larger than expected, then the registration won't be acceptable (e.g., it may be automatically rejected).

The above-described techniques may be used for the registration of color and ICGA fundus images using OCT superficial enface and choroidal layer enface images. The following is an example procedure for the registration of an ICGA fundus image to a color fundus image using OCT and/or OCTA volume data. In particular, it describes the registration of ICGA fundus to OCTA or OCT choroidal slab. A preferred method may follow the following steps:
1) Generate (OCT/OCTA) choroidal slab, such as by using Bruch's membrane and choroidal-sclera junction segmentation.
2) Generate a superficial or whole retina slab using multi retinal slab (MLS).
3) Register the ICGA fundus image to the choroidal slab (enface image) to generate an ICGA register (e.g., first registration parameters that define a first transformation for aligning the ICGA fundus image with the choroidal slab (enface image)).
4) Register OCTA superficial slab to the color fundus image (to generate second registration parameters that define a second transformation for aligning the color fundus image with the OCTA superficial slab (enface image)).
5) Use the transformation calculated at step 4 to register the ICGA register to the color fundus image.

Note that a distortion correction may be applied to the OCT/OCTA slabs prior to the registration to fundus images.

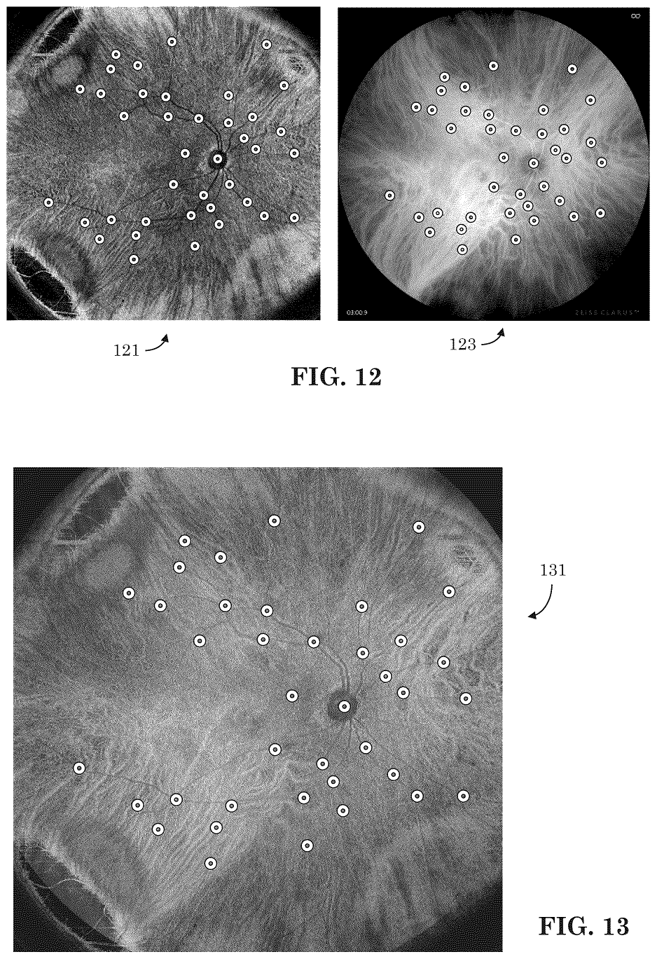

FIG. 12 shows the OCTA choroidal slab (e.g., en face image of the OCTA choroidal slab) 121 (left) and ICGA fundus image 123 (right) with overlayed matching points.

FIG. 13 shows the registered images 131 (ICGA image to choroidal slab (e.g., enface image)) overlayed on top of each other with matching points after registration.

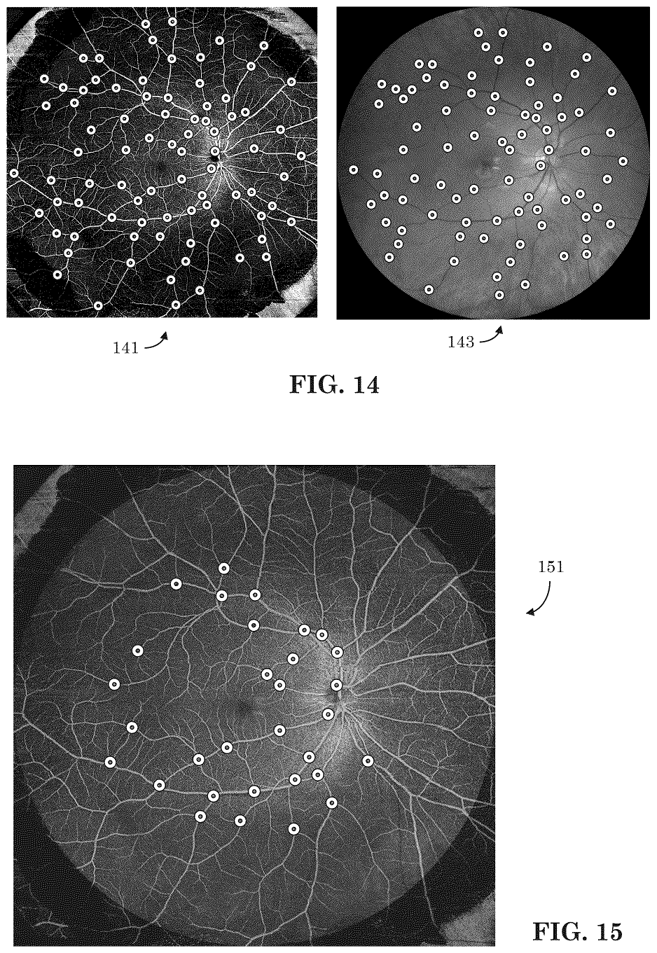

FIG. 14 shows the OCTA superficial slab 141 (en face image) (left) and color fundus image 143 (right, shown as a gray scale image) with overlayed matching points (shown as circles)

FIG. 15 shows the registered images 151 (OCTA superficial slab to color fundus) overlayed on top of each other with matching points after registration.

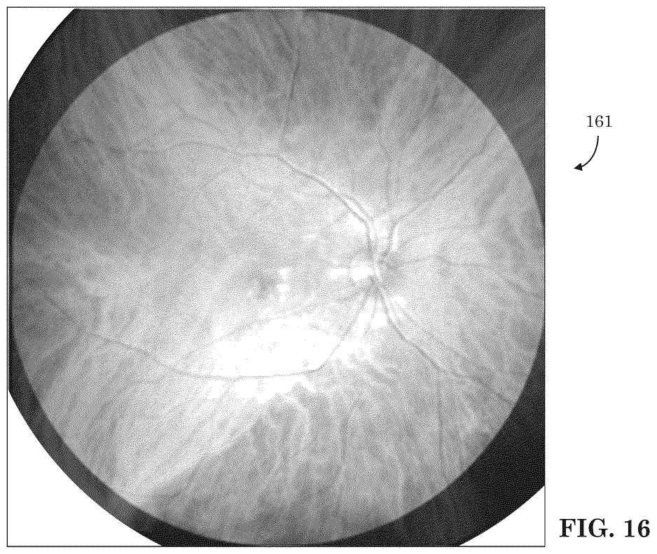

FIG. 16 shows the registered images 161 (registered ICGA image (see step 3) to the color fundus image) overlayed on top of each other.

### Fundus Imaging System

Two categories of imaging systems used to image the fundus are flood illumination imaging systems (or flood illumination imagers) and scan illumination imaging systems (or scan imagers). Flood illumination imagers flood with light an entire field of view (FOV) of interest of a specimen at the same time, such as by use of a flash lamp, and capture a full-frame image of the specimen (e.g., the fundus) with a full-frame camera (e.g., a camera having a two-dimensional (2D) photo sensor array of sufficient size to capture the desired FOV, as a whole). For example, a flood illumination fundus imager would flood the fundus of an eye with light, and capture a full-frame image of the fundus in a single image capture sequence of the camera. A scan imager provides a scan beam that is scanned across a subject, e.g., an eye, and the scan beam is imaged at different scan positions as it is scanned across the subject creating a series of image-segments that may be reconstructed, e.g., montaged, to create a composite image of the desired FOV. The scan beam could be a point, a line, or a two-dimensional area such a slit or broad line. Examples of fundus imagers are provided in U.S. Pat. No. 8,967,806 and 8,998,411.

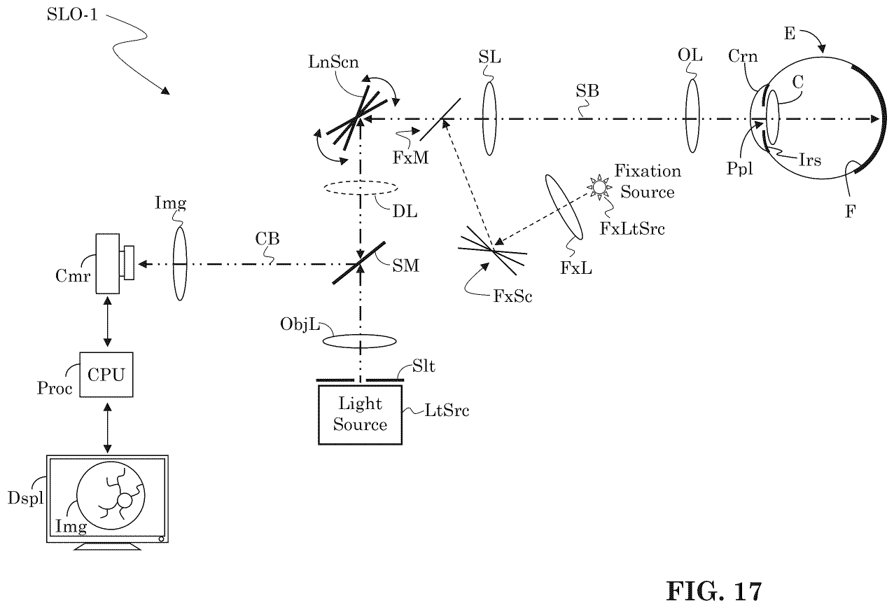

FIG. 17 illustrates an example of a slit scanning ophthalmic system SLO-1 for imaging a fundus F, which is the interior surface of an eye E opposite the eye lens (or crystalline lens) CL and may include the retina, optic disc, macula, fovea, and posterior pole. In the present example, the imaging system is in a so-called “scan-descan” configuration, wherein a scanning line beam SB traverses the optical components of the eye E (including the cornea Crn, iris Irs, pupil Ppl, and crystalline lens CL) to be scanned across the fundus F. In the case of a flood fundus imager, no scanner is needed, and the light is applied across the entire, desired field of view (FOV) at once. Other scanning configurations are known in the art, and the specific scanning configuration may not be critical. As depicted, the imaging system includes one or more light sources LtSrc, preferably a multi-color LED system or a laser system in which the etendue has been suitably adjusted. An optional slit Slt (adjustable or static) is positioned in front of the light source LtSrc and may be used to adjust the width of the scanning line beam SB. Additionally, slit Slt may remain static during imaging or may be adjusted to different widths to allow for different confocality levels and different applications either for a particular scan or during the scan for use in suppressing reflexes. An optional objective lens ObjL may be placed in front of the slit Slt. The objective lens ObjL can be any one of state-of-the-art lenses including but not limited to refractive, diffractive, reflective, or hybrid lenses/systems. The light from slit Slt passes through a pupil splitting mirror SM and is directed towards a scanner LnScn. It is desirable to bring the scanning plane and the pupil plane as near together as possible to reduce vignetting in the system. Optional optics DL may be included to manipulate the optical distance between the images of the two components. Pupil splitting mirror SM may pass an illumination beam from light source LtSrc to scanner LnScn, and reflect a detection beam from scanner LnScn (e.g., reflected light returning from eye E) toward a camera Cmr. A task of the pupil splitting mirror SM is to split the illumination and detection beams and to aid in the suppression of system reflexes. The scanner LnScn could be a rotating galvo scanner or other types of scanners (e.g., piezo or voice coil, micro-electromechanical system (MEMS) scanners, electro-optical deflectors, and/or rotating polygon scanners). Depending on whether the pupil splitting is done before or after the scanner LnScn, the scanning could be broken into two steps wherein one scanner is in an illumination path and a separate scanner is in a detection path. Specific pupil splitting arrangements are described in detail in U.S. Pat. No. 9,456,746, which is herein incorporated in its entirety by reference.

From the scanner LnScn, the illumination beam passes through one or more optics, in this case a scanning lens SL and an ophthalmic or ocular lens OL, that allow for the pupil of the eye E to be imaged to an image pupil of the system. Generally, the scan lens SL receives a scanning illumination beam from the scanner LnScn at any of multiple scan angles (incident angles), and together with Ophthalmic lens OL ensures that the beam's focal point (or focal line) moves linearly and consistently across a flat imaging plane. In the present example, ophthalmic lens OL focuses the scanning line beam SB onto the fundus F (or retina) of eye E to image the fundus. In this manner, scanning line beam SB creates a traversing scan line that travels across the fundus F. One possible configuration for these optics is a Kepler type telescope wherein the distance between the two lenses is selected to create an approximately telecentric intermediate fundus image (4-f configuration). The ophthalmic lens OL could be a single lens, an achromatic lens, or an arrangement of different lenses. All lenses could be refractive, diffractive, reflective or hybrid as known to one skilled in the art. The focal length(s) of the ophthalmic lens OL, scan lens SL and the size and/or form of the pupil splitting mirror SM and scanner LnScn could be different depending on the desired field of view (FOV), and so an arrangement in which multiple components can be switched in and out of the beam path, for example by using a flip in optic, a motorized wheel, or a detachable optical element, depending on the field of view can be envisioned. Since the field of view change results in a different beam size on the pupil, the pupil splitting can also be changed in conjunction with the change to the FOV. For example, a 45° to 60° field of view is a typical, or standard, FOV for fundus cameras. Higher fields of view, e.g., a widefield FOV, of 60°-120°, or more, may also be feasible. A widefield FOV may be desired for a combination of the Broad-Line Fundus Imager (BLFI) with another imaging modalities such as optical coherence tomography (OCT). The upper limit for the field of view may be determined by the accessible working distance in combination with the physiological conditions around the human eye. Because a typical human retina has a FOV of 140° horizontal and 80°-100° vertical, it may be desirable to have an asymmetrical field of view for the highest possible FOV on the system.

The scanning line beam SB passes through the pupil Ppl of the eye E and is directed towards the retinal, or fundus, surface F. The scanner LnScn1 adjusts the location of the light on the retina, or fundus, F such that a range of transverse locations on the eye E are illuminated. Reflected or scattered light (or emitted light in the case of fluorescence imaging) is directed back along as similar path as the illumination to define a collection beam CB on a detection path to camera Cmr.

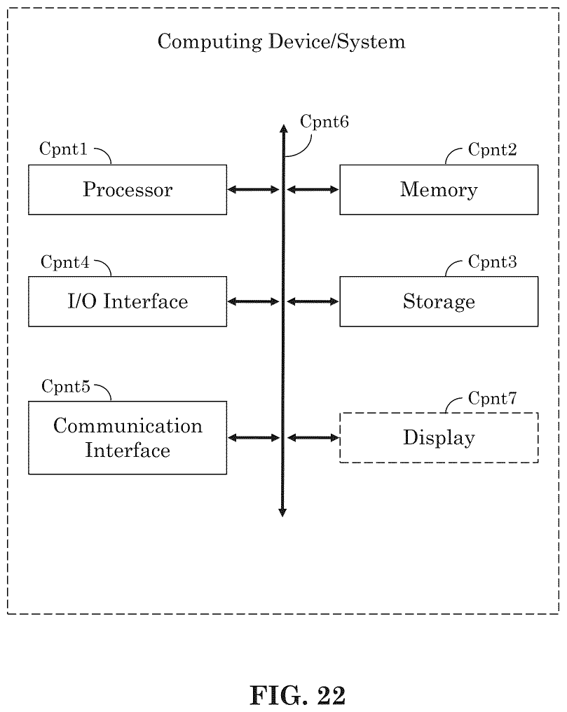

In the “scan-descan” configuration of the present, exemplary slit scanning ophthalmic system SLO-1, light returning from the eye E is “descanned” by scanner LnScn on its way to pupil splitting mirror SM. That is, scanner LnScn scans the illumination beam from pupil splitting mirror SM to define the scanning illumination beam SB across eye E, but since scanner LnScn also receives returning light from eye E at the same scan position, scanner LnScn has the effect of descanning the returning light (e.g., cancelling the scanning action) to define a non-scanning (e.g., steady or stationary) collection beam from scanner LnScn to pupil splitting mirror SM, which folds the collection beam toward camera Cmr. At the pupil splitting mirror SM, the reflected light (or emitted light in the case of fluorescence imaging) is separated from the illumination light onto the detection path directed towards camera Cmr, which may be a digital camera having a photo sensor to capture an image. An imaging (e.g., objective) lens ImgL may be positioned in the detection path to image the fundus to the camera Cmr. As is the case for objective lens ObjL, imaging lens ImgL may be any type of lens known in the art (e.g., refractive, diffractive, reflective or hybrid lens). Additional operational details, in particular, ways to reduce artifacts in images, are described in PCT Publication No. WO2016/124644, the contents of which are herein incorporated in their entirety by reference. The camera Cmr captures the received image, e.g., it creates an image file, which can be further processed by one or more (electronic) processors or computing devices (e.g., the computer system of FIG. 22). Thus, the collection beam (returning from all scan positions of the scanning line beam SB) is collected by the camera Cmr, and a full-frame image Img may be constructed from a composite of the individually captured collection beams, such as by montaging. However, other scanning configuration are also contemplated, including ones where the illumination beam is scanned across the eye E and the collection beam is scanned across a photo sensor array of the camera. PCT Publication WO 2012/059236 and US Patent Publication No. 2015/0131050, herein incorporated by reference, describe several embodiments of slit scanning ophthalmoscopes including various designs where the returning light is swept across the camera's photo sensor array and where the returning light is not swept across the camera's photo sensor array.

In the present example, the camera Cmr is connected to a processor (e.g., processing module) Proc and a display (e.g., displaying module, computer screen, electronic screen, etc.) Dspl, both of which can be part of the image system itself, or may be part of separate, dedicated processing and/or displaying unit(s), such as a computer system wherein data is passed from the camera Cmr to the computer system over a cable or computer network including wireless networks. The display and processor can be an all in one unit. The display can be a traditional electronic display/screen or of the touch screen type and can include a user interface for displaying information to and receiving information from an instrument operator, or user. The user can interact with the display using any type of user input device as known in the art including, but not limited to, mouse, knobs, buttons, pointer, and touch screen.

It may be desirable for a patient's gaze to remain fixed while imaging is carried out. One way to achieve this is to provide a fixation target that the patient can be directed to stare at. Fixation targets can be internal or external to the instrument depending on what area of the eye is to be imaged. One embodiment of an internal fixation target is shown in FIG. 17. In addition to the primary light source LtSrc used for imaging, a second optional light source FxLtSrc, such as one or more LEDs, can be positioned such that a light pattern is imaged to the retina using lens FxL, scanning element FxScn and reflector/mirror FxM. Fixation scanner FxScn can move the position of the light pattern and reflector FxM directs the light pattern from fixation scanner FxScn to the fundus F of eye E. Preferably, fixation scanner FxScn is position such that it is located at the pupil plane of the system so that the light pattern on the retina/fundus can be moved depending on the desired fixation location.

Slit-scanning ophthalmoscope systems are capable of operating in different imaging modes depending on the light source and wavelength selective filtering elements employed. True color reflectance imaging (imaging similar to that observed by the clinician when examining the eye using a hand-held or slit lamp ophthalmoscope) can be achieved when imaging the eye with a sequence of colored LEDs (red, blue, and green). Images of each color can be built up in steps with each LED turned on at each scanning position or each color image can be taken in its entirety separately. The three, color images can be combined to display the true color image, or they can be displayed individually to highlight different features of the retina. The red channel best highlights the choroid, the green channel highlights the retina, and the blue channel highlights the anterior retinal layers. Additionally, light at specific frequencies (e.g., individual colored LEDs or lasers) can be used to excite different fluorophores in the eye (e.g., autofluorescence) and the resulting fluorescence can be detected by filtering out the excitation wavelength.

The fundus imaging system can also provide an infrared reflectance image, such as by using an infrared laser (or other infrared light source). The infrared (IR) mode is advantageous in that the eye is not sensitive to the IR wavelengths. This may permit a user to continuously take images without disturbing the eye (e.g., in a preview/alignment mode) to aid the user during alignment of the instrument. Also, the IR wavelengths have increased penetration through tissue and may provide improved visualization of choroidal structures. In addition, fluorescein angiography (FA) and indocyanine green (ICG) angiography imaging can be accomplished by collecting images after a fluorescent dye has been injected into the subject's bloodstream. For example, in FA (and/or ICG) a series of time-lapse images may be captured after injecting a light-reactive dye (e.g., fluorescent dye) into a subject's bloodstream. It is noted that care should be taken since the fluorescent dye may lead to a life-threatening allergic reaction in a portion of the population. High contrast, greyscale images are captured using specific light frequencies selected to excite the dye. As the dye flows through the eye, various portions of the eye are made to glow brightly (e.g., fluoresce), making it possible to discern the progress of the dye, and hence the blood flow, through the eye.

### Optical Coherence Tomography Imaging System

Generally, optical coherence tomography (OCT) uses low-coherence light to produce two-dimensional (2D) and three-dimensional (3D) internal views of biological tissue. OCT enables in vivo imaging of retinal structures. OCT angiography (OCTA) produces flow information, such as vascular flow from within the retina. Examples of OCT systems are provided in U.S. Pat. Nos. 6,741,359 and 9,706,915, and examples of an OCTA systems may be found in U.S. Pat. Nos. 9,700,206 and 9,759,544, all of which are herein incorporated in their entirety by reference. An exemplary OCT/OCTA system is provided herein.

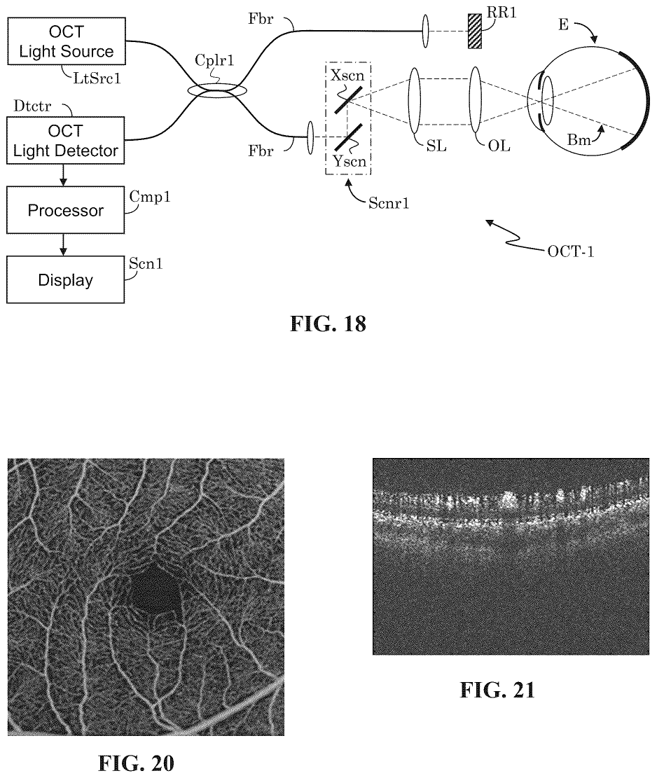

FIG. 18 illustrates a generalized frequency domain optical coherence tomography (FD-OCT) system used to collect 3D image data of the eye, in accordance with various embodiments. An FD-OCT system OCT_1 includes a light source, LtSrc1. Typical light sources include, but are not limited to, broadband light sources with short temporal coherence lengths or swept laser sources. A beam of light from light source LtSrc1 is routed, typically by optical fiber Fbr1, to illuminate a sample, e.g., eye E; a typical sample being tissues in the human eye. The light source LrSrc1 may, for example, be a broadband light source with short temporal coherence length in the case of spectral domain OCT (SD-OCT) or a wavelength tunable laser source in the case of swept source OCT (SS-OCT). The light may be scanned, typically with a scanner Scnr1 between the output of the optical fiber Fbr1 and the sample E, so that the beam of light (dashed line Bm) is scanned laterally over the region of the sample to be imaged. The light beam from scanner Scnr1 may pass through a scan lens SL and an ophthalmic lens OL and be focused onto the sample E being imaged. The scan lens SL (or telecentric scan lens) is used to scan a light beam across the sample. The scan lens helps ensure the light beam's focal point (or focal line) moves linearly and consistently across a flat imaging plane. Together with the ophthalmic lens OL, the light beam is focused onto the sample. The present example illustrates a scan beam that is scanned in two lateral directions (e.g., in x and y directions on a Cartesian plane) to scan a desired field of view (FOV). An example of this would be a point-field OCT, which uses a point-field beam to scan across a sample. Consequently, scanner Scnr1 is illustratively shown to include two sub-scanner: a first sub-scanner Xscn for scanning the point-field beam across the sample in a first direction (e.g., a horizontal x-direction); and a second sub-scanner Yscn for scanning the point-field beam on the sample in traversing second direction (e.g., a vertical y-direction). If the scan beam were a line-field beam (e.g., a line-field OCT), which may sample an entire line-portion of the sample at a time, then only one scanner scans the line-field beam across the sample to span the desired FOV. If the scan beam were a full-field beam (e.g., a full-field OCT), no scanner may be needed, and the full-field light beam may be applied across the entire, desired FOV at once.

Irrespective of the type of beam used, light scattered from the sample (e.g., sample light) is collected. In the present example, scattered light returning from the sample is collected into the same optical fiber Fbr1 used to route the light for illumination. Reference light derived from the same light source LtSrc1 travels a separate path, in this case involving optical fiber Fbr2 and retro-reflector RR1 with an adjustable optical delay. Those skilled in the art will recognize that a transmissive reference path can also be used and that the adjustable delay could be placed in the sample or reference arm of the interferometer. Collected sample light is combined with reference light, for example, in a fiber coupler Cplr1, to form light interference in an OCT light detector Dtctr1 (e.g., photodetector array, digital camera, etc.). Although a single fiber port is shown going to the detector Dtctr1, those skilled in the art will recognize that various designs of interferometers can be used for balanced or unbalanced detection of the interference signal. The output from the detector Dtctr1is supplied to a processor (e.g., internal or external computing device) Cmp1 that converts the observed interference into depth information of the sample. The depth information may be stored in a memory associated with the processor Cmp1 and/or displayed on a display (e.g., computer/electronic display/screen) Scn1. The processing and storing functions may be localized within the OCT instrument, or functions may be offloaded onto (e.g., performed on) an external processor (e.g., an external computing device), to which the collected data may be transferred. An example of a computing device (or computer system) is shown in FIG. 22. This unit could be dedicated to data processing or perform other tasks which are quite general and not dedicated to the OCT device. The processor (computing device) Cmp1 may include, for example, a field-programmable gate array (FPGA), a digital signal processor (DSP), an application specific integrated circuit (ASIC), a graphics processing unit (GPU), a system on chip (SoC), a central processing unit (CPU), a general purpose graphics processing unit (GPGPU), or a combination thereof, that may performs some, or the entire, processing steps in a serial and/or parallelized fashion with one or more host processors and/or one or more external computing devices.

The sample and reference arms in the interferometer could consist of bulk-optics, fiber-optics, or hybrid bulk-optic systems and could have different architectures such as Michelson, Mach-Zehnder or common-path based designs as would be known by those skilled in the art. Light beam as used herein should be interpreted as any carefully directed light path. Instead of mechanically scanning the beam, a field of light can illuminate a one or two-dimensional area of the retina to generate the OCT data (see for example, U.S. Pat. No. 9,332,902; D. Hillmann et al, “Holoscopy—Holographic Optical Coherence Tomography,” Optics Letters, 36(13): 2390 2011; Y. Nakamura, et al, “High-Speed Three Dimensional Human Retinal Imaging by Line Field Spectral Domain Optical Coherence Tomography,” Optics Express, 15(12):7103 2007; Blazkiewicz et al, “Signal-To-Noise Ratio Study of Full-Field Fourier-Domain Optical Coherence Tomography,” Applied Optics, 44(36):7722 (2005)). In time-domain systems, the reference arm has a tunable optical delay to generate interference. Balanced detection systems are typically used in TD-OCT and SS-OCT systems, while spectrometers are used at the detection port for SD-OCT systems. The systems and methods could be applied to any type of OCT system. Various aspects could apply to any type of OCT system or other types of ophthalmic diagnostic systems and/or multiple ophthalmic diagnostic systems including but not limited to fundus imaging systems, visual field test devices, and scanning laser polarimeters.

In Fourier Domain optical coherence tomography (FD-OCT), each measurement is the real-valued spectral interferogram (Sj(k)). The real-valued spectral data typically goes through several post-processing steps including background subtraction, dispersion correction, etc. The Fourier transform of the processed interferogram, results in a complex valued OCT signal output Aj(z)=|Aj|eiφ. The absolute value of this complex OCT signal, |Aj|, reveals the profile of scattering intensities at different path lengths, and therefore scattering as a function of depth (z-direction) in the sample. Similarly, the phase, φj can also be extracted from the complex valued OCT signal. The profile of scattering as a function of depth is called an axial scan (A-scan). A set of A-scans measured at neighboring locations in the sample produces a cross-sectional image (tomogram or B-scan) of the sample. A collection of B-scans collected at different transverse locations on the sample makes up a data volume or cube. For a particular volume of data, the term fast axis refers to the scan direction along a single B-scan whereas slow axis refers to the axis along which multiple B-scans are collected. The term “cluster scan” may refer to a single unit or block of data generated by repeated acquisitions at the same (or substantially the same) location (or region) for the purposes of analyzing motion contrast, which may be used to identify blood flow. A cluster scan can consist of multiple A-scans or B-scans collected with relatively short time separations at approximately the same location(s) on the sample. Since the scans in a cluster scan are of the same region, static structures remain relatively unchanged from scan to scan within the cluster scan, whereas motion contrast between the scans that meets predefined criteria may be identified as blood flow.

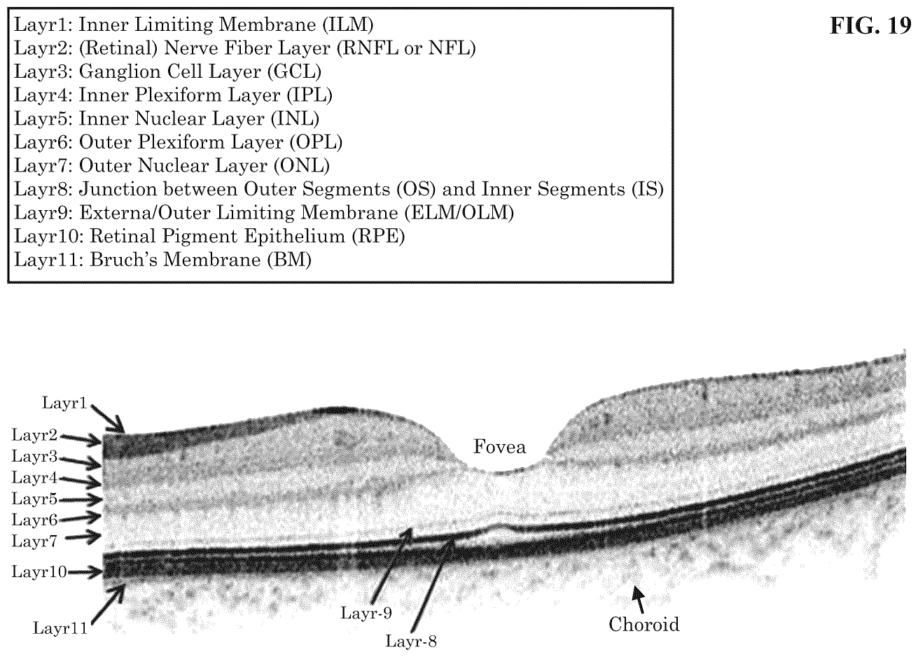

A variety of ways to create B-scans are known in the art including but not limited to: along the horizontal or x-direction, along the vertical or y-direction, along the diagonal of x and y, or in a circular or spiral pattern. B-scans may be in the x-z dimensions but may be any cross-sectional image that includes the z-dimension. An example OCT B-scan image of a normal retina of a human eye is illustrated in FIG. 19. An OCT B-scan of the retinal provides a view of the structure of retinal tissue. For illustration purposes, FIG. 19 identifies various canonical retinal layers and layer boundaries. The identified retinal boundary layers include (from top to bottom): the inner limiting membrane (ILM) Lyer1, the retinal nerve fiber layer (RNFL or NFL) Layr2, the ganglion cell layer (GCL) Layr3, the inner plexiform layer (IPL) Layr4, the inner nuclear layer (INL) Layr5, the outer plexiform layer (OPL) Layr6, the outer nuclear layer (ONL) Layr7, the junction between the outer segments (OS) and inner segments (IS) (indicated by reference character Layr8) of the photoreceptors, the external or outer limiting membrane (ELM or OLM) Layr9, the retinal pigment epithelium (RPE) Layr10, and the Bruch's membrane (BM) Layr11.

In OCT Angiography, or Functional OCT, analysis algorithms may be applied to OCT data collected at the same, or approximately the same, sample locations on a sample at different times (e.g., a cluster scan) to analyze motion or flow (see for example US Patent Publication Nos. 2005/0171438, 2012/0307014, 2010/0027857, 2012/0277579 and U.S. Pat. No. 6,549,801, all of which are herein incorporated in their entirety by reference). An OCT system may use any one of a number of OCT angiography processing algorithms (e.g., motion contrast algorithms) to identify blood flow. For example, motion contrast algorithms can be applied to the intensity information derived from the image data (intensity-based algorithm), the phase information from the image data (phase-based algorithm), or the complex image data (complex-based algorithm). An en face image is a 2D projection of 3D OCT data (e.g., by averaging the intensity of each individual A-scan, such that each A-scan defines a pixel in the 2D projection). Similarly, an en face vasculature image is an image displaying motion contrast signal in which the data dimension corresponding to depth (e.g., z-direction along an A-scan) is displayed as a single representative value (e.g., a pixel in a 2D projection image), typically by summing or integrating all or an isolated portion of the data (see for example U.S. Pat. No. 7,301,644 herein incorporated in its entirety by reference). OCT systems that provide an angiography imaging functionality may be termed OCT angiography (OCTA) systems.

FIG. 20 shows an example of an en face vasculature image. After processing the data to highlight motion contrast using any of the motion contrast techniques known in the art, a range of pixels corresponding to a given tissue depth from the surface of internal limiting membrane (ILM) in retina, may be summed to generate the en face (e.g., frontal view) image of the vasculature. FIG. 21 shows an exemplary B-scan of a vasculature (OCTA) image. As illustrated, structural information may not be well-defined since blood flow may traverse multiple retinal layers making them less defined than in a structural OCT B-scan, as shown in FIG. 19. Nonetheless, OCTA provides a non-invasive technique for imaging the microvasculature of the retina and the choroid, which may be critical to diagnosing and/or monitoring various pathologies. For example, OCTA may be used to identify diabetic retinopathy by identifying microaneurysms, neovascular complexes, and quantifying foveal avascular zone and nonperfused areas. Moreover, OCTA has been shown to be in good agreement with fluorescein angiography (FA), a more traditional, but more evasive, technique including the injection of a dye to observe vascular flow in the retina. Additionally, in dry age-related macular degeneration, OCTA has been used to monitor a general decrease in choriocapillaris flow. Similarly in wet age-related macular degeneration, OCTA can provides a qualitative and quantitative analysis of choroidal neovascular membranes. OCTA has also been used to study vascular occlusions, e.g., evaluation of nonperfused areas and the integrity of superficial and deep plexus.

### Fundus Imaging System

Two categories of imaging systems used to image the fundus are flood illumination imaging systems (or flood illumination imagers) and scan illumination imaging systems (or scan imagers). Flood illumination imagers flood with light an entire field of view (FOV) of interest of a specimen at the same time, such as by use of a flash lamp, and capture a full-frame image of the specimen (e.g., the fundus) with a full-frame camera (e.g., a camera having a two-dimensional (2D) photo sensor array of sufficient size to capture the desired FOV, as a whole). For example, a flood illumination fundus imager would flood the fundus of an eye with light, and capture a full-frame image of the fundus in a single image capture sequence of the camera. A scan imager provides a scan beam that is scanned across a subject, e.g., an eye, and the scan beam is imaged at different scan positions as it is scanned across the subject creating a series of image-segments that may be reconstructed, e.g., montaged, to create a composite image of the desired FOV. The scan beam could be a point, a line, or a two-dimensional area such a slit or broad line. Examples of fundus imagers are provided in U.S. Pat. Nos. 8,967,806 and 8,998,411.

FIG. 17 illustrates an example of a slit scanning ophthalmic system SLO-1 for imaging a fundus F, which is the interior surface of an eye E opposite the eye lens (or crystalline lens) CL and may include the retina, optic disc, macula, fovea, and posterior pole. In the present example, the imaging system is in a so-called “scan-descan” configuration, wherein a scanning line beam SB traverses the optical components of the eye E (including the cornea Crn, iris Irs, pupil Ppl, and crystalline lens CL) to be scanned across the fundus F. In the case of a flood fundus imager, no scanner is needed, and the light is applied across the entire, desired field of view (FOV) at once. Other scanning configurations are known in the art, and the specific scanning configuration may not be critical. As depicted, the imaging system includes one or more light sources LtSrc, preferably a multi-color LED system or a laser system in which the etendue has been suitably adjusted. An optional slit Slt (adjustable or static) is positioned in front of the light source LtSrc and may be used to adjust the width of the scanning line beam SB. Additionally, slit Slt may remain static during imaging or may be adjusted to different widths to allow for different confocality levels and different applications either for a particular scan or during the scan for use in suppressing reflexes. An optional objective lens ObjL may be placed in front of the slit Slt. The objective lens ObjL can be any one of state-of-the-art lenses including but not limited to refractive, diffractive, reflective, or hybrid lenses/systems. The light from slit Slt passes through a pupil splitting mirror SM and is directed towards a scanner LnScn. It is desirable to bring the scanning plane and the pupil plane as near together as possible to reduce vignetting in the system. Optional optics DL may be included to manipulate the optical distance between the images of the two components. Pupil splitting mirror SM may pass an illumination beam from light source LtSrc to scanner LnScn, and reflect a detection beam from scanner LnScn (e.g., reflected light returning from eye E) toward a camera Cmr. A task of the pupil splitting mirror SM is to split the illumination and detection beams and to aid in the suppression of system reflexes. The scanner LnScn could be a rotating galvo scanner or other types of scanners (e.g., piezo or voice coil, micro-electromechanical system (MEMS) scanners, electro-optical deflectors, and/or rotating polygon scanners). Depending on whether the pupil splitting is done before or after the scanner LnScn, the scanning could be broken into two steps wherein one scanner is in an illumination path and a separate scanner is in a detection path. Specific pupil splitting arrangements are described in detail in U.S. Pat. No. 9,456,746, which is herein incorporated in its entirety by reference.

From the scanner LnScn, the illumination beam passes through one or more optics, in this case a scanning lens SL and an ophthalmic or ocular lens OL, that allow for the pupil of the eye E to be imaged to an image pupil of the system. Generally, the scan lens SL receives a scanning illumination beam from the scanner LnScn at any of multiple scan angles (incident angles), and together with Ophthalmic lens OL ensures that the beam's focal point (or focal line) moves linearly and consistently across a flat imaging plane. In the present example, ophthalmic lens OL focuses the scanning line beam SB onto the fundus F (or retina) of eye E to image the fundus. In this manner, scanning line beam SB creates a traversing scan line that travels across the fundus F. One possible configuration for these optics is a Kepler type telescope wherein the distance between the two lenses is selected to create an approximately telecentric intermediate fundus image (4-f configuration). The ophthalmic lens OL could be a single lens, an achromatic lens, or an arrangement of different lenses. All lenses could be refractive, diffractive, reflective or hybrid as known to one skilled in the art. The focal length(s) of the ophthalmic lens OL, scan lens SL and the size and/or form of the pupil splitting mirror SM and scanner LnScn could be different depending on the desired field of view (FOV), and so an arrangement in which multiple components can be switched in and out of the beam path, for example by using a flip in optic, a motorized wheel, or a detachable optical element, depending on the field of view can be envisioned. Since the field of view change results in a different beam size on the pupil, the pupil splitting can also be changed in conjunction with the change to the FOV. For example, a 45° to 60° field of view is a typical, or standard, FOV for fundus cameras. Higher fields of view, e.g., a widefield FOV, of 60°-120°, or more, may also be feasible. A widefield FOV may be desired for a combination of the Broad-Line Fundus Imager (BLFI) with another imaging modalities such as optical coherence tomography (OCT). The upper limit for the field of view may be determined by the accessible working distance in combination with the physiological conditions around the human eye. Because a typical human retina has a FOV of 140° horizontal and 80°-100° vertical, it may be desirable to have an asymmetrical field of view for the highest possible FOV on the system.

The scanning line beam SB passes through the pupil Ppl of the eye E and is directed towards the retinal, or fundus, surface F. The scanner LnScn1 adjusts the location of the light on the retina, or fundus, F such that a range of transverse locations on the eye E are illuminated. Reflected or scattered light (or emitted light in the case of fluorescence imaging) is directed back along as similar path as the illumination to define a collection beam CB on a detection path to camera Cmr.

In the “scan-descan” configuration of the present, exemplary slit scanning ophthalmic system SLO-1, light returning from the eye E is “descanned” by scanner LnScn on its way to pupil splitting mirror SM. That is, scanner LnScn scans the illumination beam from pupil splitting mirror SM to define the scanning illumination beam SB across eye E, but since scanner LnScn also receives returning light from eye E at the same scan position, scanner LnScn has the effect of descanning the returning light (e.g., cancelling the scanning action) to define a non-scanning (e.g., steady or stationary) collection beam from scanner LnScn to pupil splitting mirror SM, which folds the collection beam toward camera Cmr. At the pupil splitting mirror SM, the reflected light (or emitted light in the case of fluorescence imaging) is separated from the illumination light onto the detection path directed towards camera Cmr, which may be a digital camera having a photo sensor to capture an image. An imaging (e.g., objective) lens ImgL may be positioned in the detection path to image the fundus to the camera Cmr. As is the case for objective lens ObjL, imaging lens ImgL may be any type of lens known in the art (e.g., refractive, diffractive, reflective or hybrid lens). Additional operational details, in particular, ways to reduce artifacts in images, are described in PCT Publication No. WO2016/124644, the contents of which are herein incorporated in their entirety by reference. The camera Cmr captures the received image, e.g., it creates an image file, which can be further processed by one or more (electronic) processors or computing devices (e.g., the computer system of FIG. 22). Thus, the collection beam (returning from all scan positions of the scanning line beam SB) is collected by the camera Cmr, and a full-frame image Img may be constructed from a composite of the individually captured collection beams, such as by montaging. However, other scanning configuration are also contemplated, including ones where the illumination beam is scanned across the eye E and the collection beam is scanned across a photo sensor array of the camera. PCT Publication WO 2012/059236 and US Patent Publication No. 2015/0131050, herein incorporated by reference, describe several embodiments of slit scanning ophthalmoscopes including various designs where the returning light is swept across the camera's photo sensor array and where the returning light is not swept across the camera's photo sensor array.

In the present example, the camera Cmr is connected to a processor (e.g., processing module) Proc and a display (e.g., displaying module, computer screen, electronic screen, etc.) Dspl, both of which can be part of the image system itself, or may be part of separate, dedicated processing and/or displaying unit(s), such as a computer system wherein data is passed from the camera Cmr to the computer system over a cable or computer network including wireless networks. The display and processor can be an all in one unit. The display can be a traditional electronic display/screen or of the touch screen type and can include a user interface for displaying information to and receiving information from an instrument operator, or user. The user can interact with the display using any type of user input device as known in the art including, but not limited to, mouse, knobs, buttons, pointer, and touch screen.

It may be desirable for a patient's gaze to remain fixed while imaging is carried out. One way to achieve this is to provide a fixation target that the patient can be directed to stare at. Fixation targets can be internal or external to the instrument depending on what area of the eye is to be imaged. One embodiment of an internal fixation target is shown in FIG. 17. In addition to the primary light source LtSrc used for imaging, a second optional light source FxLtSrc, such as one or more LEDs, can be positioned such that a light pattern is imaged to the retina using lens FxL, scanning element FxScn and reflector/mirror FxM. Fixation scanner FxScn can move the position of the light pattern and reflector FxM directs the light pattern from fixation scanner FxScn to the fundus F of eye E. Preferably, fixation scanner FxScn is position such that it is located at the pupil plane of the system so that the light pattern on the retina/fundus can be moved depending on the desired fixation location.

Slit-scanning ophthalmoscope systems are capable of operating in different imaging modes depending on the light source and wavelength selective filtering elements employed. True color reflectance imaging (imaging similar to that observed by the clinician when examining the eye using a hand-held or slit lamp ophthalmoscope) can be achieved when imaging the eye with a sequence of colored LEDs (red, blue, and green). Images of each color can be built up in steps with each LED turned on at each scanning position or each color image can be taken in its entirety separately. The three, color images can be combined to display the true color image, or they can be displayed individually to highlight different features of the retina. The red channel best highlights the choroid, the green channel highlights the retina, and the blue channel highlights the anterior retinal layers. Additionally, light at specific frequencies (e.g., individual colored LEDs or lasers) can be used to excite different fluorophores in the eye (e.g., autofluorescence) and the resulting fluorescence can be detected by filtering out the excitation wavelength.

The fundus imaging system can also provide an infrared reflectance image, such as by using an infrared laser (or other infrared light source). The infrared (IR) mode is advantageous in that the eye is not sensitive to the IR wavelengths. This may permit a user to continuously take images without disturbing the eye (e.g., in a preview/alignment mode) to aid the user during alignment of the instrument. Also, the IR wavelengths have increased penetration through tissue and may provide improved visualization of choroidal structures. In addition, fluorescein angiography (FA) and indocyanine green (ICG) angiography imaging can be accomplished by collecting images after a fluorescent dye has been injected into the subject's bloodstream. For example, in FA (and/or ICG) a series of time-lapse images may be captured after injecting a light-reactive dye (e.g., fluorescent dye) into a subject's bloodstream. It is noted that care should be taken since the fluorescent dye may lead to a life-threatening allergic reaction in a portion of the population. High contrast, greyscale images are captured using specific light frequencies selected to excite the dye. As the dye flows through the eye, various portions of the eye are made to glow brightly (e.g., fluoresce), making it possible to discern the progress of the dye, and hence the blood flow, through the eye.

### Optical Coherence Tomography Imaging System

Generally, optical coherence tomography (OCT) uses low-coherence light to produce two-dimensional (2D) and three-dimensional (3D) internal views of biological tissue. OCT enables in vivo imaging of retinal structures. OCT angiography (OCTA) produces flow information, such as vascular flow from within the retina. Examples of OCT systems are provided in U.S. Pat. Nos. 6,741,359 and 9,706,915, and examples of an OCTA systems may be found in U.S. Pat. Nos. 9,700,206 and 9,759,544, all of which are herein incorporated in their entirety by reference. An exemplary OCT/OCTA system is provided herein.

FIG. 18 illustrates a generalized frequency domain optical coherence tomography (FD-OCT) system used to collect 3D image data of the eye suitable for use with various embodiments. An FD-OCT system OCT_1 includes a light source, LtSrc1. Typical light sources include, but are not limited to, broadband light sources with short temporal coherence lengths or swept laser sources. A beam of light from light source LtSrc1 is routed, typically by optical fiber Fbr1, to illuminate a sample, e.g., eye E; a typical sample being tissues in the human eye. The light source LrSrc1 may, for example, be a broadband light source with short temporal coherence length in the case of spectral domain OCT (SD-OCT) or a wavelength tunable laser source in the case of swept source OCT (SS-OCT). The light may be scanned, typically with a scanner Scnr1 between the output of the optical fiber Fbr1 and the sample E, so that the beam of light (dashed line Bm) is scanned laterally over the region of the sample to be imaged. The light beam from scanner Scnr1 may pass through a scan lens SL and an ophthalmic lens OL and be focused onto the sample E being imaged. The scan lens SL (or telecentric scan lens) is used to scan a light beam across the sample. The scan lens helps ensure the light beam's focal point (or focal line) moves linearly and consistently across a flat imaging plane. Together with the ophthalmic lens OL, the light beam is focused onto the sample. The present example illustrates a scan beam that is scanned in two lateral directions (e.g., in x and y directions on a Cartesian plane) to scan a desired field of view (FOV). An example of this would be a point-field OCT, which uses a point-field beam to scan across a sample. Consequently, scanner Scnr1 is illustratively shown to include two sub-scanner: a first sub-scanner Xscn for scanning the point-field beam across the sample in a first direction (e.g., a horizontal x-direction); and a second sub-scanner Yscn for scanning the point-field beam on the sample in traversing second direction (e.g., a vertical y-direction). If the scan beam were a line-field beam (e.g., a line-field OCT), which may sample an entire line-portion of the sample at a time, then only one scanner is used to scan the line-field beam across the sample to span the desired FOV. If the scan beam were a full-field beam (e.g., a full-field OCT), no scanner may be needed, and the full-field light beam may be applied across the entire, desired FOV at once.

Irrespective of the type of beam used, light scattered from the sample (e.g., sample light) is collected. In the present example, scattered light returning from the sample is collected into the same optical fiber Fbr1 used to route the light for illumination. Reference light derived from the same light source LtSrc1 travels a separate path, in this case involving optical fiber Fbr2 and retro-reflector RR1 with an adjustable optical delay. Those skilled in the art will recognize that a transmissive reference path can also be used and that the adjustable delay could be placed in the sample or reference arm of the interferometer. Collected sample light is combined with reference light, for example, in a fiber coupler Cplr1, to form light interference in an OCT light detector Dtctr1 (e.g., photodetector array, digital camera, etc.). Although a single fiber port is shown going to the detector Dtctr1, those skilled in the art will recognize that various designs of interferometers can be used for balanced or unbalanced detection of the interference signal. The output from the detector Dtctr1is supplied to a processor (e.g., internal or external computing device) Cmp1 that converts the observed interference into depth information of the sample. The depth information may be stored in a memory associated with the processor Cmp1 and/or displayed on a display (e.g., computer/electronic display/screen) Scn1. The processing and storing functions may be localized within the OCT instrument, or functions may be offloaded onto (e.g., performed on) an external processor (e.g., an external computing device), to which the collected data may be transferred. An example of a computing device (or computer system) is shown in FIG. 22. This unit could be dedicated to data processing or perform other tasks which are quite general and not dedicated to the OCT device. The processor (computing device) Cmp1 may include, for example, a field-programmable gate array (FPGA), a digital signal processor (DSP), an application specific integrated circuit (ASIC), a graphics processing unit (GPU), a system on chip (SoC), a central processing unit (CPU), a general purpose graphics processing unit (GPGPU), or a combination thereof, that may performs some, or the entire, processing steps in a serial and/or parallelized fashion with one or more host processors and/or one or more external computing devices.

The sample and reference arms in the interferometer could consist of bulk-optics, fiber-optics, or hybrid bulk-optic systems and could have different architectures such as Michelson, Mach-Zehnder or common-path based designs as would be known by those skilled in the art. Light beam as used herein should be interpreted as any carefully directed light path. Instead of mechanically scanning the beam, a field of light can illuminate a one or two-dimensional area of the retina to generate the OCT data (see for example, U.S. Pat. No. 9,332,902; D. Hillmann et al, “Holoscopy—Holographic Optical Coherence Tomography,” Optics Letters, 36(13): 2390 2011; Y. Nakamura, et al, “High-Speed Three Dimensional Human Retinal Imaging by Line Field Spectral Domain Optical Coherence Tomography,” Optics Express, 15(12):7103 2007; Blazkiewicz et al, “Signal-To-Noise Ratio Study of Full-Field Fourier-Domain Optical Coherence Tomography,” Applied Optics, 44(36):7722 (2005)). In time-domain systems, the reference arm has a tunable optical delay to generate interference. Balanced detection systems are typically used in TD-OCT and SS-OCT systems, while spectrometers are used at the detection port for SD-OCT systems. The systems and methods could be applied to any type of OCT system. Various embodiments could apply to any type of OCT system or other types of ophthalmic diagnostic systems and/or multiple ophthalmic diagnostic systems including but not limited to fundus imaging systems, visual field test devices, and scanning laser polarimeters.

In Fourier Domain optical coherence tomography (FD-OCT), each measurement is the real-valued spectral interferogram (Sj(k)). The real-valued spectral data typically goes through several post-processing steps including background subtraction, dispersion correction, etc. The Fourier transform of the processed interferogram, results in a complex valued OCT signal output Aj(z)=|Aj|eiφ. The absolute value of this complex OCT signal, |Aj|, reveals the profile of scattering intensities at different path lengths, and therefore scattering as a function of depth (z-direction) in the sample. Similarly, the phase, φj can also be extracted from the complex valued OCT signal. The profile of scattering as a function of depth is called an axial scan (A-scan). A set of A-scans measured at neighboring locations in the sample produces a cross-sectional image (tomogram or B-scan) of the sample. A collection of B-scans collected at different transverse locations on the sample makes up a data volume or cube. For a particular volume of data, the term fast axis refers to the scan direction along a single B-scan whereas slow axis refers to the axis along which multiple B-scans are collected. The term “cluster scan” may refer to a single unit or block of data generated by repeated acquisitions at the same (or substantially the same) location (or region) for the purposes of analyzing motion contrast, which may be used to identify blood flow. A cluster scan can consist of multiple A-scans or B-scans collected with relatively short time separations at approximately the same location(s) on the sample. Since the scans in a cluster scan are of the same region, static structures remain relatively unchanged from scan to scan within the cluster scan, whereas motion contrast between the scans that meets predefined criteria may be identified as blood flow.

A variety of ways to create B-scans are known in the art including but not limited to: along the horizontal or x-direction, along the vertical or y-direction, along the diagonal of x and y, or in a circular or spiral pattern. B-scans may be in the x-z dimensions but may be any cross-sectional image that includes the z-dimension. An example OCT B-scan image of a normal retina of a human eye is illustrated in FIG. 19. An OCT B-scan of the retinal provides a view of the structure of retinal tissue. For illustration purposes, FIG. 19 identifies various canonical retinal layers and layer boundaries. The identified retinal boundary layers include (from top to bottom): the inner limiting membrane (ILM) Lyer1, the retinal nerve fiber layer (RNFL or NFL) Layr2, the ganglion cell layer (GCL) Layr3, the inner plexiform layer (IPL) Layr4, the inner nuclear layer (INL) Layr5, the outer plexiform layer (OPL) Layr6, the outer nuclear layer (ONL) Layr7, the junction between the outer segments (OS) and inner segments (IS) (indicated by reference character Layr8) of the photoreceptors, the external or outer limiting membrane (ELM or OLM) Layr9, the retinal pigment epithelium (RPE) Layr10, and the Bruch's membrane (BM) Layr11.

In OCT Angiography, or Functional OCT, analysis algorithms may be applied to OCT data collected at the same, or approximately the same, sample locations on a sample at different times (e.g., a cluster scan) to analyze motion or flow (see for example US Patent Publication Nos. 2005/0171438, 2012/0307014, 2010/0027857, 2012/0277579 and U.S. Pat. No. 6,549,801, all of which are herein incorporated in their entirety by reference). An OCT system may use any one of a number of OCT angiography processing algorithms (e.g., motion contrast algorithms) to identify blood flow. For example, motion contrast algorithms can be applied to the intensity information derived from the image data (intensity-based algorithm), the phase information from the image data (phase-based algorithm), or the complex image data (complex-based algorithm). An en face image is a 2D projection of 3D OCT data (e.g., by averaging the intensity of each individual A-scan, such that each A-scan defines a pixel in the 2D projection). Similarly, an en face vasculature image is an image displaying motion contrast signal in which the data dimension corresponding to depth (e.g., z-direction along an A-scan) is displayed as a single representative value (e.g., a pixel in a 2D projection image), typically by summing or integrating all or an isolated portion of the data (see for example U.S. Pat. No. 7,301,644 herein incorporated in its entirety by reference). OCT systems that provide an angiography imaging functionality may be termed OCT angiography (OCTA) systems.

FIG. 20 shows an example of an en face vasculature image. After processing the data to highlight motion contrast using any of the motion contrast techniques known in the art, a range of pixels corresponding to a given tissue depth from the surface of internal limiting membrane (ILM) in retina, may be summed to generate the en face (e.g., frontal view) image of the vasculature. FIG. 21 shows an exemplary B-scan of a vasculature (OCTA) image. As illustrated, structural information may not be well-defined since blood flow may traverse multiple retinal layers making them less defined than in a structural OCT B-scan, as shown in FIG. 19. Nonetheless, OCTA provides a non-invasive technique for imaging the microvasculature of the retina and the choroid, which may be critical to diagnosing and/or monitoring various pathologies. For example, OCTA may be used to identify diabetic retinopathy by identifying microaneurysms, neovascular complexes, and quantifying foveal avascular zone and nonperfused areas. Moreover, OCTA has been shown to be in good agreement with fluorescein angiography (FA), a more traditional, but more evasive, technique including the injection of a dye to observe vascular flow in the retina. Additionally, in dry age-related macular degeneration, OCTA has been used to monitor a general decrease in choriocapillaris flow. Similarly in wet age-related macular degeneration, OCTA can provides a qualitative and quantitative analysis of choroidal neovascular membranes. OCTA has also been used to study vascular occlusions, e.g., evaluation of nonperfused areas and the integrity of superficial and deep plexus.

1. A method of registering a first fundus image of an eye to a second fundus image of
the eye, comprising:
accessing, by one or more processors, the first fundus image, the first fundus image being of a first imaging modality type; accessing, by the one or more processors, the second fundus image, the second fundus image being of a second imaging modality type different than the first imaging modality type;
accessing, by the one or more processors, an optical coherence tomography (OCT) volume scan of the eye;
generating, by the one or more processors, a first enface image of a first sub-volume of the OCT volume scan;
generating, by the one or more processors, a second enface image of a second sub-volume of the OCT volume scan, the second sub-volume being different than the first sub-volume;
registering, by the one or more processors, the first fundus image to the first enface image;
registering, by the one or more processors, the second fundus image to the second enface image to define a transformation; and
registering, by the one or more processors, the first fundus image to the second fundus image using the transformation.

2. The method of claim 1, wherein:
the first enface image is an OCT structural enface image; and
the second face image is an OCT angiography enface image.

3. The method of claim 1, wherein:
the first sub-volume is a choroidal slab; and
the second sub-volume is one of a superficial retinal slab, superficial plus deeper retinal slap, or whole retina slab.

4. The method of claim 3, wherein:
the first fundus image is an indocyanine green angiography (ICGA) image; and
the second fundus image is a color fundus image.

5. The method of claim 4, wherein:
the first enface image is an OCT structural enface image; and
the second face image is an OCT angiography enface image.

6. A method of registering a first fundus image of an eye to a second fundus image of the eye, comprising:
accessing, by one or more processors, the first fundus image, the first fundus image being of a first imaging modality type;
accessing, by the one or more processors, the second fundus image, the second fundus image being comprised of a plurality of modality channels, each different than the first modality type;
separately, by the one or more processors, registering the first fundus image to each of the different modality channels of the second fundus image to identify separately corresponding sets of matched landmarks;
combining, by the one or more processors, select matched landmarks from each of the different sets of matched landmarks to define an aggregate of matched landmarks; and
registering, by the one or more processors, the first fundus image to the second fundus image using the aggregate of matched landmarks.

7. The method of claim 6, wherein the first fundus image is one of an infrared (IR) fundus image, single color fundus image, or angiography fundus image.

8. The method of claim 7, wherein the second fundus image is a color fundus image comprising of a plurality of color channels, each color channel being a separate one of the plurality of modality channels.

9. A method of registering a first fundus image of an eye to a second fundus image of the eye, comprising:
designate, by the one or more processors, the first fundus image a reference image;
designate, by the one or more processors, the second fundus image a distorted image;
identify, by the one or more processors, a plurality of landmarks in in the reference image; and
repeat, by the one or more processors, the following two steps until a predefined successful level of successful registration is achieved:
a) apply a projection transformation to the distorted image based on currently matched landmarks between the distorted image and the reference image; and
b) update the projection transformation based on the current state of the distorted image and the currently matched landmarks between the distorted image and the reference image.

10. The method of claim 9, wherein:
step (a) uses a current projective transformation matrix T and an identify matrix; and
in step (b), an interim projective transformation matrix T0 is defined based on currently identified matched landmarks, and current matrix T is update by multiplying to the interim projective transformation T0.

11. The method of claim 9, wherein the landmarks in the reference image are identified by random sampling the reference image or by use a feature finding algorithm.

12. The method of claim 9, wherein the predefined successful level of successful registration is achieved when an optimization is converged.
